(19) United States
(12) Patent Application Publication
(10) Pub. No.: US 2004/0097860 A1
(43) Pub. Date: May 20, 2004

(54) Wrap for a horse's leg and method for wrapping and treatment thereof

(76) Inventor: Brady J. Tauber, Mt. Prospect, IL (US)

Correspondence Address: MCCRACKEN & FRANK LLP, 200 W. ADAMS STREET, SUITE 2150, CHICAGO, IL, 60606, US

(21) Appl. No.: 10/300,235

(22) Filed: Nov. 20, 2002

(51) Int. Cl. A61L 15/00

(52) U.S. Cl. 602/75

(57) ABSTRACT

A wrap for a horse's leg includes a wicking layer adjacent a thermal layer, a protective layer adjacent the thermal layer and a fasteners attached to the protective layer. A method for wrapping a horse's leg includes the steps of wrapping the horse's leg with a composite having a wicking layer, a thermal layer and a protective layer and securing the composite to the horse's leg. A method for treating a horse's leg includes applying a preparation to the leg and wrapping the horse's leg with a composite comprising a wicking layer, a thermal layer, and a protective layer.

### TECHNICAL FIELD

The present invention relates generally to a wrap for a horse's leg, and more particularly to a method for wrapping and treating the horse's leg.

### BACKGROUND ART

There are several circumstances under which the need to wrap a horse's lower leg between the hoof and the knee may arise. For example, a wrap may be used during transport when a horse is forced to stand in a motor vehicle for an extended period of time, after periods of strenuous exercise, or during the sweat process commonly used to reduce injury due to inflammation and swelling.

The sweat process includes the application of at least three layers of various materials in multiple steps. First, any one of several preparations including dimethylsulfoxide, nitrofurazone ointment, petroleum jelly, glycerin, glycerol, epsom salts, and mineral oil is applied to a dry horse's leg. Next, a first layer of cotton batting, or a cotton/poly fill pillow is wrapped around the leg. The first layer comprising cotton batting, or cotton/poly fill pillow is surrounded with a second layer of cellophane or Saran Wrap®. The application of the cotton batting, or cotton/poly fill pillow and Saran Wrap® or cellophane is reversible such that the cellophane or Saran Wrap® layer may be applied first and surrounded by the cotton, batting, or cotton/poly fill pillow. In either case, a bandage is applied over the first and second layer of materials as a third and final layer.

The process of individually applying three separate layers is difficult for a horse owner due to the continuous movement of the horse's leg throughout the application of each layer and in some cases can be detrimental to the horse's leg. For example, misapplication of the Saran Wrap® or cellophane layer when applied directly to the horse's leg can cause a decrease in circulation to the leg or cording which may constrict the tendons. Furthermore, application of the cotton batting or cotton/poly fill pillow as the first layer directly applied to the horse's leg may become damp and therefore lose its thermal properties. This may cause a decrease in the temperature of the leg in cooler or cold climates thus counteracting the purpose of the sweat process altogether.

Freeman U.S. Pat. No. 3,338,028 discloses a spiral self-adhering track bandage for racehorses. The track bandage includes a single layer of knitted material with releasable attachment means secured thereto.

Surgical and animal bandaging is disclosed by Moon U.S. Pat. Nos. 3,504,672 and 3,667,462. The bandaging is a seamless tube comprised of synthetic yarn having a Velcro® hook strap at one end and a second end wherein the Velcro® hook strap may be secured to either a Velcro® nap strip or directly to the tube itself. The purpose of the bandage, which exhibits elasticity and stretch in all directions, is to provide shock absorption.

Fossel U.S. Pat. No. 3,880,161 discloses an elastic bandage and fastener system for the bandage. The bandage is a single layer of elastic sheet material and the fastener comprises an elongated fastener tape capable of being wrapped around the elastic bandage. The fastener maintains the bandage in place and provides a mechanism for increasing and decreasing the tension created by the bandage.

Porner U.S. Pat. No. 4,099,269 discloses lightweight protective cover for the limbs of humans and the fetlocks of horses. The protective device includes an outer covering and at least one layer of suitable sheet material having a plurality of spaced apart hollow pockets. The outer covering can be vinyl, plastic, and leather. The purpose of the inner layer of sheet material is to provide protective cushioning by absorbing shock.

A knee bandage is disclosed by Yovankin U.S. Pat. No. 4,424,809. The knee bandage is comprised of an inner and outer layer of the same stretch fabric stitched together to form a plurality of elongated pockets wherein each pocket receives a pad of foam plastic. The bandage is disclosed as a protective covering and can be used as a post surgical bandage, as a cover for a leg wherein the leg contains poultice plaster or sweat, as knee protection during shipment of an animal, or as a cool pack to create a cooling sensation for reducing pain and inflammation.

Shapiro U.S. Pat. No. 4,538,602 discloses a leg wrap for an animal including an inner and outer layer of any suitable material with fastening means. The outer end of the inner layer is joined to the inner end of the outer layer. The inner layer is wrapped around the leg of a horse and the outer layer is wrapped around the inner layer and then fastened.

### SUMMARY OF THE INVENTION

In accordance with one aspect of the present invention, a wrap for a horse's leg comprises a wicking layer, a thermal layer adjacent the wicking layer, a protective layer adjacent the thermal layer and attached to the wicking layer and the thermal layer, and a fastener attached to the protective layer.

According to a further aspect of the present invention, a method for wrapping a horse's leg includes the steps of wrapping the horse's leg with a composite having a wicking layer, a thermal layer adjacent the wicking layer and a protective layer adjacent the thermal layer and securing the composite to the horse's leg.

According to a still further aspect of the present invention, a method for treating a horse's leg includes applying a preparation to the horse's leg and wrapping the horse's leg with a composite comprising a wicking layer, a thermal batting, and protective layer, and securing the composite in place.

According to a yet further aspect of the present invention, a wrap for a horse's leg comprises a composite formed from wicking layer and a thermal layer, a protective flap attached to one end of the composite; wherein the protective flap has a length and a width sufficient to substantially cover the composite as the composite and the protective flap are wrapped around the horse's leg; and a fastener attached to the protective flap.

Other aspects and advantages of the present invention will become apparent upon consideration of the following detailed description.

### BRIEF DESCRIPTION OF THE DRAWINGS

FIG. 1 is a front view of a wrap for a horse's leg;

FIG. 2 is a sectional view taken generally along the line 2-2 of FIG. 1;

FIG. 3 is an exploded isometric view of the wrap of FIG. 1;

FIG. 4 is a pictorial view of FIG. 1 wrapped around a horse's leg;

FIG. 5 is a front view similar to FIG. 1 of a second embodiment of a wrap;

FIG. 6 is a fragmentary view of FIG. 5 showing an alternative fastener;

FIG. 7 is an exploded isometric view of a third embodiment of a wrap; and

FIG. 8 is pictorial view of a fourth embodiment of a wrap secured with tape around a horse's leg.

### DETAILED DESCRIPTION OF THE PREFERRED EMBODIMENTS

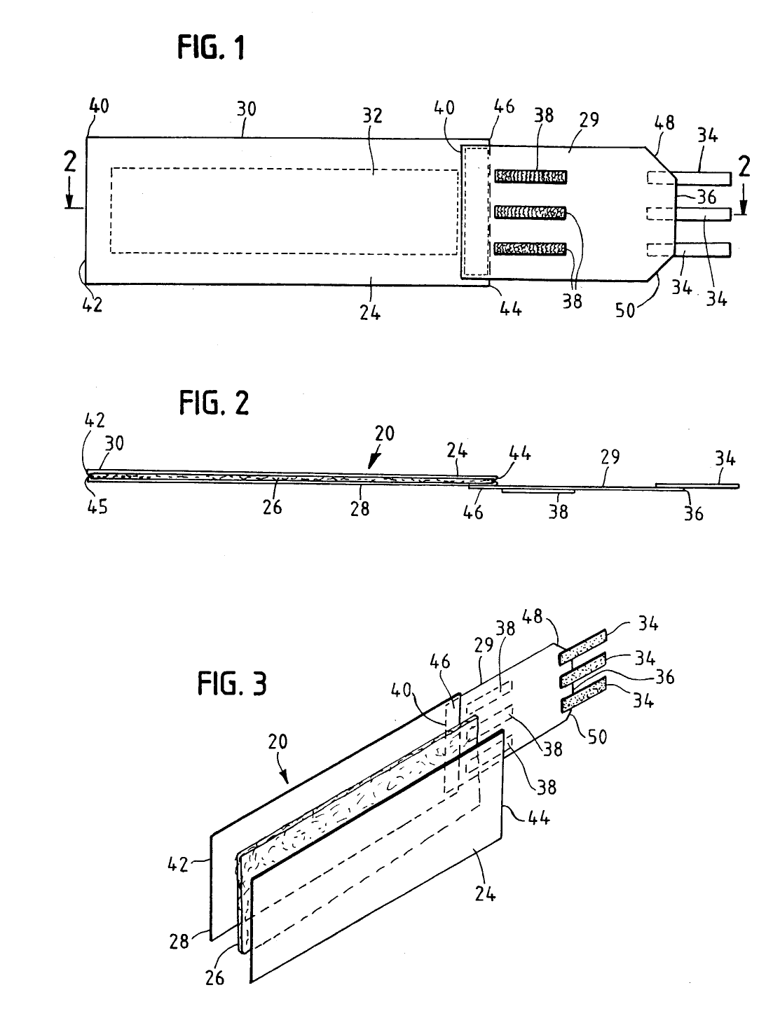

Referring now to FIGS. 1-4, a first embodiment of a wrap 20 for a horse's leg 22 includes three layers. A wicking layer 24 of the wrap 20 is formed from a wicking fabric and is to be placed against the horse's leg 22. The wicking layer 24 transports moisture from the horse's leg 22 into a thermal layer 26. The thermal layer 26 of the wrap 20 is formed from a thermal batting and placed adjacent the wicking layer 24 to provide warmth and moisture protection to the horse's leg 22. The moisture absorbed from the horse's leg 22 is held within the thermal layer 26. The wicking layer 24 prevents the horse's leg 22 from directly contacting the thermal layer 26 and any absorbed moisture contained within the thermal layer 26. A protective layer 28 of the wrap 20 is formed from a material that provides an exterior covering to protect the wrap 20 and the horse's leg 22 from the elements. Preferably the protective layer 28 is formed from a material that stretches in at least one dimension. In addition, the protective layer 28 allows the wrap 20 to be snuggly placed about the horse's leg 22 thereby preventing injury from securing the wrap 20 too tightly around the horse's leg. The wicking layer 24, the thermal layer 26 and the protective layer 28 are joined together to form a composite 30, having a first end 42 and a second end 44. The thickness of the composite 30 may vary from one quarter of an inch to three quarters of an inch with one half inch in thickness being preferred.

The assembly of the composite 30 includes the following steps. The wicking layer 24 is joined to the thermal layer 26 that is joined to the protective layer 28. Any method of joining the three layers together to form the composite 30 may be employed, however, an interior seam 45 on the outside of the three layers 24, 26 and 28 forming soft edges is preferred. This preferred construction reduces irritation to the horse's leg 22 that may be caused by a sharp edge around the periphery of the wrap 20. In addition to the interior seam 45, it is preferred but not essential to include a rectangular shaped stitched center portion 32 that joins all three layers 24, 26 and 28 for additional support. A protective flap 29 preferably formed from the same material as the protective layer 28 is attached to the second end 44 of the composite 30 by sewing, adhesive bonding, thermal bonding or any other suitable attachment method. In addition, the protective flap 29 can be a continuation or extension of the protective layer 28 as shown in FIG. 5.

A series of fastening strips 34, such as Velcro® hook strips, are attached to the distal end 36 of the protective flap 29. Even though three fastening strips 34 are shown, any number of fastening strips 34 can be used. In addition, a corresponding number of nap pads 38, such as Velcro® loop pads, are attached on the exterior of the protective flap 29 near a distal end 40 of the protective flap 29. If the protective flap 29 is shorter than the circumference of the horse's leg 22, then in this case the nap pads 38 can be attached directly to the exterior surface of the protective layer 28. Alternatively, a single nap pad 66 extending transversely across the wrap 20 may be substituted in place of the multiple nap pads 38 as shown in the embodiment of FIG. 5.

The wicking layer 24 is generally rectangular in shape having a length ranging from about eight to about thirty eight inches, preferably between about twenty to about thirty six inches, and has a width that varies from about twelve to about twenty four inches depending on the size of the horse's leg as measured from the hoof to the knee. Suitable materials for use as the wicking layer 24 include any woven or non-woven fabric capable of transporting sweat or moisture from the horse's leg to the thermal layer thereby keeping the horse's leg 22 dry. If moisture is trapped against the horse's leg 22, the moisture may chill the horse's leg 22, which may decrease the flow of blood to the leg thereby counteracting the entire reason for using a leg wrap. Examples of suitable wicking fabrics include Dryline® from Miliken®, Akwatek® and Akwadyne® from Comfort Technologies and Thermastat and Coolmax® polyester available from DuPont. The preferred material for the wicking layer 24 is Coolmax®.

The thermal layer 26 is also rectangular in shape and of a similar size to the wicking layer 24. Suitable materials for the thermal layer 26 include any lightweight, mid-loft, breathable woven or non-woven fabric having moisture vapor transportation properties and thermal insulation properties. Suitable thermal materials include: cotton/poly fill; Thermolite® from Dupont, which is a thin insulation having a hollow polyester fiber laid in random layers with an acrylic binder (loose felted) needle punched through the cross section to attach layers and tie them down, such as Thermolite® extreme, Thermoloft®, Thermolite® micro, Thermolite® extra or Thermolite® plus, etc.; Quallofil® from DuPont; Hollofil® from DuPont; Thinsulate insulation available from 3M, which is a blend of polyester and polyolefin fibers; Primaloft microfiber insulation available from Albany International; Aerozip polyester insulation available from Toray; Polarguard continuous filament fiber insulation available from Hoechst Celanese; Polartec insulation available from Malden Mills; Thermalon olefin insulation with polyester batting available from Thermalon Industries; Thermore polyolefin/polyester/resin blend insulation available from FISI/Concept III; Trevira Loft nine denier polyester staple fiber insulation available from Hoechst Celanese; and Ultrafibre reflected metal plated fiber insulation available from Ultrafibre, Inc. The preferred thermal material is Primaloft. The thermal layer 26 is attached to the first layer of 24 by any conventional attachment technique, including sewing, adhesive bonding, thermal bonding and the like.

The protective layer 28 is formed from any breathable, abrasion resistant fabric. Preferably these fabrics should stretch in at least one direction and most preferably these fabrics should stretch in both directions. In addition, these fabrics should also offer protection from adverse weather conditions such as snow, wind, or rain. Suitable materials include Gortex®, Lycra®, Spandura®, Cordura®, Cordura® Lycra® blend, and blends thereof. The preferred material for the protective layer is a Cordura® Lycra® blend. In the embodiment as shown in FIGS. 1-4, the protective layer 28 is rectangular in shape and the approximately the same size as the wicking layer 24 and the thermal layer 26. The protective layer 28 is placed adjacent to the thermal layer 26 so that these three layers can be attached to each other using any of the attachment methods described above to form the composite 30.

As seen in FIG. 3, the protective flap 29 is attached to the protective layer 28 and includes two inward tapers 48 and 50 at the distal end 40 of the protective flap 29. The inward tapers 48 and 50 minimize binding that may restrict circulation in the horse's leg. A rectangular shaped end portion 46 of the protective flap 29 is placed over the second end 42 of the composite 30 and attached thereto by any suitable attachment method as described above and preferably by stitching through all three layers of the composite 30. The protective flap 29 can be any length and width to enable the wrap 20 to be secured around the horse's leg 22. It is preferred that the protective flap 29 has a width from about one inch narrower than the composite 30 to about the same width as the composite 30 and a length of between about two to about twenty inches and most preferably about sixteen inches. The protective flap 29 can be rectangular, can include the inward tapers 48 and 50 as shown in FIG. 3 or can include a taper the entire length of the protective flap 29 as shown in FIG. 5.

FIG. 5 illustrates a second embodiment of a wrap 52 wherein a protective layer 54 includes an integral protective flap 56 that is formed from the same piece of material as the protective layer 54. The assembly of the wrap 52 is similar to the assembly of the wrap 20, however, the combined thermal layer 26 and wicking layer 24 are attached to the integral protective layer 54 in a region extending from the first end 42 to a line 58 to form a three layer composite similar to the composite 30. The protective flap 56 of the integral protective layer 54 performs in a manner similar to the protective flap 29. As shown in FIG. 5, sides 60 and 62 of the protective flap slope inwardly from the line 58 to a distal end 64 of the protective flap 56. This tapering minimizes the possibility of the wrap 52 restricting circulation in the horse's leg 22. A series of fasteners 34 are attached at the distal end 64 of the protective flap 56. The wrap 52 includes three fasteners 34 and the single nap pad 66. The size of the composite area of FIG. 5 and the protective flap 56 can be the same as described relative to similar components in the embodiment of FIG. 1.

FIG. 6 shows a partial view of the protective flap 56 of FIG. 5 with a different reclosable fastener system. As shown in FIG. 5, three straps 90, each having a series of holes 94, are attached to the distal end 64 of the proactive flap 56. The straps 90 are sized so that each strap 90 engages a buckle 92 that is secured to the protective flap 56 by a suitable attachment method. The straps 90 may be formed from any suitable material that will not stretch significantly, such as natural or synthetic leather, flexible plastic and the like. Similarly, the buckles 92 may be formed from any suitable material such as metal or plastic.

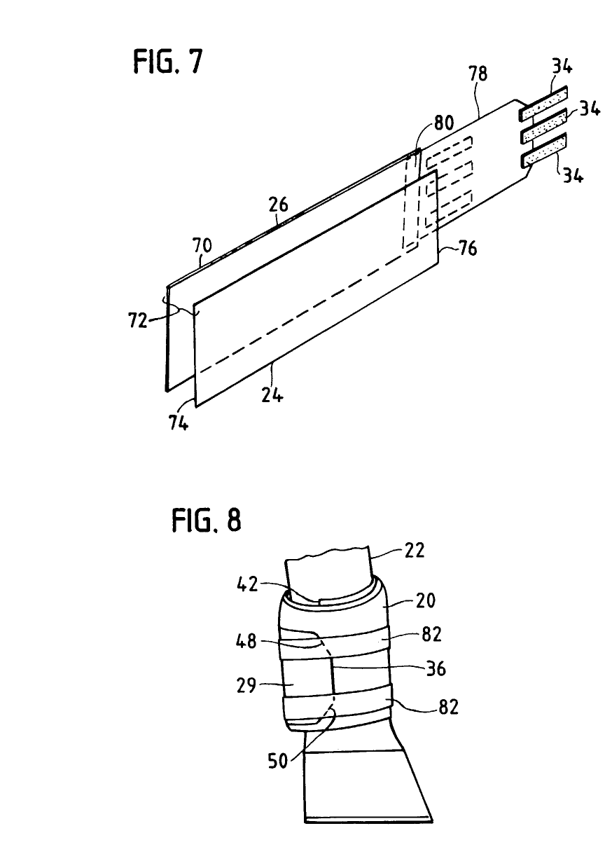

FIG. 7 illustrates a third embodiment of a wrap 70 that has a composite 72 formed from the wicking layer 24 and the thermal layer 26. The composite 72 has a first end 74 and a second end 76. A protective flap 78 is attached directly to the thermal layer 26 of the composite 72 at an overlap zone 80. The composite 72 is wrapped around the horse's leg until the protective flap 78 is reached. Then the protective flap 78 is wrapped around the composite 72 and secured in place by fasteners 34. The dimensions of the protective flap 78 are such that when the protective flap 78 is wrapped around the composite 72, the protective flap 78 substantially covers the composite 72 to form a layer of protection around the wrap 70 and the horse's leg 22. The preferred length for the protective flap 78 is between about eight to about twenty inches. The composite 72 can have a length of from about eight to about thirty eight inches, preferably between about twenty to about thirty six inches, and a width of from about twelve to about twenty four inches depending on the size of the horse's leg as measured from the hoof to the knee.

As shown in FIG. 8, the wrap 20 may be held in place around the horse's leg 22 by two strips of a tape 82 such as surgical tape, masking tape, duct tape, vet wrap and similar tapes. Other suitable attachment mechanisms include, safety pins, ace bandages, elasticon and similar materials.

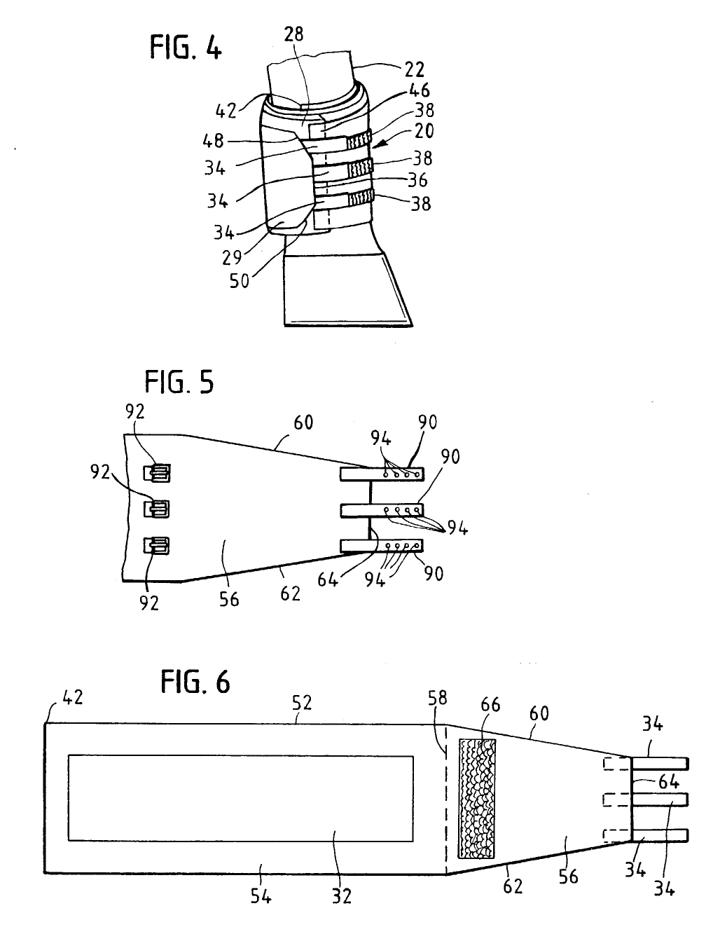

A method for wrapping a horse's leg 22 after strenuous exercise, during the sweat process, or for warmth in the winter includes the following steps. As seen in FIG. 4, the first end 42 of the composite 30 is held in place with the wicking layer 24 facing the horse's leg 22. The composite 30 is continuously wrapped around the horse's leg and the protective flap 29 is wrapped around the composite 30. Since the protective flap 29 preferably is formed from material that will stretch in at least one direction, the composite 30 can be firmly secured around the horse's leg 22. Once the end of the protective flap 29 is reached, the fasteners 34 and 38 are attached to each other to secure the wrap 20 to the horse's leg 22. Typically, the composite 30 is wrapped around the horse's leg 22 three times so that the completed wrap 20 includes three layers of the composite 30 plus one layer of the protective flap 29.

A method of treatment for the horse's leg 22 includes the following steps. First, a commercial and/or homemade preparation including but not limited to dimethylsulfoxide, nitrofurazone ointment, petroleum jelly, glycerin, glycerol, epsom salts, and mineral oil is applied directly to the horse's leg 22. Second, the wrap 20 is applied to the horse's leg 22 for a period of 12 hours and then removed for 12 hours. The process of treating the horse's leg 22 includes repeating steps one and two until the wound is healed.

Numerous modifications to the present invention will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications which come within the scope of the appended claims are reserved.

We claim:

1. A wrap for a horse's leg, comprising:
a wicking layer;
a thermal layer adjacent the wicking layer
a protective layer adjacent the thermal layer and attached to the wicking layer and the thermal layer; and
a fastener attached to the protective layer.

2. The wrap of claim 1, wherein a protective flap is attached to the protective layer.

3. The wrap of claim 1 wherein an interior stitch attaches the wicking layer, thermal layer and protective layer thereby forming soft edges around the exterior of the wicking layer and the protective layer to protect the horse's limbs.

4. The wrap of claim 1 wherein the fastener comprises nap and hook portions.

5. The wrap of claim 1 wherein the wicking layer is a woven fabric capable of transporting sweat from the horse's leg to the wicking fabric.

6. The wrap of claim 1 wherein the wicking layer is a non-woven fabric capable of transporting sweat from the horse's leg to the wicking fabric.

7. The wrap of claim 1 wherein the wicking layer is capable of evaporating sweat.

8. The wrap of claim 1 wherein the wicking layer is capable of air permeability in a wet or dry state.

9. The wrap of claim 1 wherein the wicking layer is formed from Coolmax®.

10. The wrap of claim 1 wherein the thermal layer has moisture vapor transportation properties.

11. The wrap of claim 1 wherein the thermal layer is formed from Primaloft.

12. The wrap of claim 1 wherein the protective layer is moisture resistant and wind resistant to protect the horse's leg.

13. The wrap of claim 1 wherein the protective layer is abrasion resistant.

14. The wrap of claim 1 wherein the protective layer is formed from a stretch fabric capable of expansion in at least one direction.

15. The wrap of claim 1 wherein the protective layer is formed from a stretch fabric capable of expansion in any direction.

16. The wrap of claim 1 wherein the protective layer is formed from a Cordura® Lycra® blend.

17. The wrap of claim 1 wherein the protective layer has a length greater than the wicking layer and thermal layer to form an integral protective flap.

18. The wrap of claim 2, wherein the fastener is a reclosable fastener and is attached to the protective flap.

19. The wrap of claim 18 wherein the reclosable fastener is adjustable.

20. The wrap of claim 1 wherein the wicking layer, the thermal layer and the protective layer form a composite having a rectangular shape having a length from about eight to about thirty-eight inches and a width from about twelve to about twenty-four inches.

21. A method for wrapping a horse's leg, comprising the steps of:
wrapping the horse's leg with a composite comprising a wicking layer, a thermal layer adjacent the wicking layer, and a protective layer adjacent the thermal layer; and
securing the composite in place.

22. The method of claim 21, wherein the step of wrapping further includes the step of positioning the wicking layer against the horse's leg.

23. The method of claim 21, wherein the step of securing further includes the step of wrapping a protective flap around the composite.

24. The method of claim 21 wherein the step of securing further includes the step of fastening a reclosable fastener.

25. A method for treating a horse's leg, comprising the steps of:
applying a preparation to the horse's leg;
wrapping the horse's leg with a composite comprising a wicking layer, a thermal layer adjacent the wicking layer, and a protective layer adjacent the thermal layer; and
securing the composite in place.

26. The method of claim 25, wherein the step of securing further includes the step of wrapping a protective flap around the composite.

27. The method of claim 25 wherein the step of securing further includes the step of fastening a reclosable fastening strips.

28. The method of claim 25 wherein the step of applying further includes the step of rubbing the poltice or dimethylsulfoxide combination into the leg.

29. The method of claim 25 further including the step of increasing circulation to the area of the horse's leg covered by the composite.

30. The method of claim 25 wherein the preparation is selected from the group consisting of dimethylsulfoxide, nitrofurazone ointment, petroleum jelly, glycerin, glycerol, epsom salts, and mineral oil.

31. A wrap for a horse's leg comprising:
a composite formed from a wicking layer and a thermal layer;
a protective flap attached to one end of the composite; wherein the protective flap has a length and a width sufficient to substantially cover the composite as the composite and the protective flap are wrapped around the horse's leg; and
a fastener attached to the protective flap.

32. The wrap of claim 28 wherein the protective flap is formed from a material that will stretch in at least one dimension.

33. The wrap of claim 28 wherein the protective flap is formed from a material that is wind resistant and water resistant.

34. The wrap of claim 28 wherein the fastener is a reclosable fastener.

35. The wrap of claim 28 wherein the wicking layer is formed from Coolmax®.

36. The wrap of claim 28 wherein the thermal layer is formed from Primaloft.

37. The wrap of claim 28 wherein the protective flap is formed from a Cordura® Lycra® blend.
